(19) United States
(12) Patent Application Publication
(10) Pub. No.: US 2021/0045638 A1
(43) Pub. Date: Feb. 18, 2021

(54) TWO-IN-ONE THERMOMETER

(71) Applicants: Richard MCDUFFIE, Marlborough, MA (US); Helen of Troy Limited, St. Michael (BB)

(72) Inventors: Richard McDuffie, Worcester, MA (US); Zoey Juhng, Fort Lee, NJ (US); Lyndon T. Treacy, Brooklyn, NY (US); Mathieu Zastawny, Jersey City, NJ (US); Dio Climaco Cavero, North Merrick, NY (US); Adam Sanchez, Wantage, NJ (US); Thibault Lerailler, New York, NY (US); Theodore Kostopoulos, Hudson, MA (US)

(21) Appl. No.: 16/977,584

(22) PCT Filed: Mar. 8, 2019
(86) PCT No.: PCT/US2019/021283

§ 371 Date: Sep. 2, 2020

Related U.S. Application Data
(60) Provisional application No. 62/640,926, filed on Mar. 9, 2018.

(51) Int. Cl. A61B 5/01; A61B 5/06; A61B 5/00; G01J 5/02; G01J 5/04; G01K 1/08; G01K 13/00

(52) U.S. Cl. CPC A61B 5/01; A61B 5/06; A61B 5/6817; G01K 13/004; G01J 5/021; G01J 5/04; G01K 1/086; A61B 5/6886

(57) ABSTRACT

A cap for a thermometer having a probe configured to be placed in contact with a patients body or to be inserted into a body cavity of the patient and a temperature sensor includes a cap housing, a proximity sensor and a proximity sensor electrical conductor. The cap housing is configured to selectively connect with and to be selectively detached from the thermometer and at least partially surround the probe of the thermometer when connected with the thermometer. The proximity sensor is connected with the cap. The proximity sensor electrical conductor is connected with the cap and electrically connected with the proximity sensor. A thermometer including the cap is also provided.

### BACKGROUND

Infrared ear thermometers known in the art typically include an infrared sensor positioned inside a probe that is configured to be inserted into an ear canal aimed in the direction of the tympanic membrane. The infrared sensor and the control circuitry to which it is connected measure intensity of infrared radiation that emanates from the ear canal surface and converts the infrared radiation signal into an output temperature.

U.S. Pat. No. 8,517,603 B2 discloses an infrared thermometer capable of being used as an ear thermometer as described above and as a forehead thermometer, which measures thermal radiation from the patient's external body surface in the temporal region of the forehead. Both the aforementioned ear thermometer and forehead thermometer can be referred to as contact thermometers because a portion, typically what is referred to as a probe, contacts the patient when measuring the patient's body temperature.

While U.S. Pat. No. 8,517,603 B2 discloses a remote attachment to convert an IR ear thermometer into a remote non-contact thermometer, a Fresnel lens is used to focus incoming IR radiation toward an IR sensor in the probe

### SUMMARY

In view of the foregoing, a thermometer includes a housing, a temperature sensor, a cap, a proximity sensor, and control circuitry. The housing includes a probe configured to be placed in contact with a patient's body or to be inserted into a body cavity of the patient. The temperature sensor is positioned in the housing and is configured to detect a first signal. The cap is configured to cooperate with the housing, and is positionable in a first sensing operating position in which the cap is positioned with respect to the probe so as to inhibit insertion of the probe into the body cavity of the patient. The proximity sensor is connected with the cap and is configured to detect a second signal. The control circuitry is in electrical communication with the temperature sensor and the proximity sensor. The control circuitry is configured (1) to measure intensity of the first signal received by the temperature sensor and to convert the first signal into a temperature output that reflects a patient's body temperature, (2) to measure intensity of the second signal received by the proximity sensor and to convert the second signal into a distance output that reflects a distance between the proximity sensor and a target area on the patient's body, and (3) to switch between a first mode in which the patient's body temperature is being measured with the probe being offset from the patient's body and a second mode in which the patient's body temperature is being measured with the probe in contact with the patient's body or inserted into the body cavity of the patient.

A method of operating an infrared thermometer is also disclosed. The method includes positioning the cap in a forehead mode operating position in which the cap is positioned with respect to the probe so as to inhibit insertion of the probe into a patient's ear. With the cap positioned in the forehead mode, the method further includes pointing the temperature sensor toward the forehead of the patient and prompting the thermometer to detect a temperature signal, which is indicative of the patient's body temperature, via the temperature sensor and to detect a distance signal, which is indicative of a distance between the proximity sensor and the patient's forehead, via the proximity sensor. The method further includes positioning the cap in an in ear mode operating position in which the cap is offset from the probe so as to allow insertion of the probe into the patient's ear, and with the cap in the in ear mode, prompting the thermometer to detect another temperature signal via the temperature sensor.

### BRIEF DESCRIPTION OF THE DRAWINGS

FIG. 1 is a schematic depiction of a thermometer without a cap attached to the housing of the thermometer.

FIG. 2 is a front perspective of the thermometer with the cap attached to the housing in a first sensing operating position.

FIG. 3 is a side perspective view of the thermometer depicted in FIG. 2 with the cap removed from the housing.

FIG. 4 is a side elevation view of the thermometer with the cap attached to the housing in the first sensing operating position.

FIG. 5 is a rear elevation view of the thermometer with the cap attached to the housing in the first sensing operating position.

FIG. 6 is a side elevation view of the thermometer with the cap removed from the housing.

FIG. 7 is a rear elevation view of the thermometer with the cap removed from the housing

FIG. 8 is a rear perspective view of the thermometer with the cap removed from the housing.

FIG. 9 is a close-up front perspective view of an upper portion of the thermometer with the cap removed from the housing.

FIG. 10 is a perspective view of a thermometer with a cap in a first sensing operating position.

FIG. 11 is a perspective view of the thermometer of FIG. 10 with the cap in a second sensing operating position.

FIG. 12 is an exploded view of a portion of the thermometer shown in FIG. 10.

FIG. 13 is a cross-sectional view of an upper portion of the thermometer with the cap in a first sensing operating position.

FIG. 14 is a cross-sectional view of the upper portion of the thermometer with the cap in a second sensing operating position.

FIG. 15 is another cross-sectional view of the upper portion of the thermometer with the cap in the first sensing operating position.

FIG. 16 is another cross-sectional view of the upper portion of the thermometer with the cap in the second sensing operating position.

FIG. 16A is a close-up view of the circled portion in FIG. 16.

### DETAILED DESCRIPTION

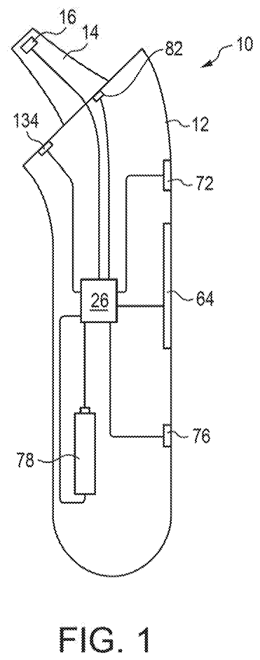

FIG. 1 schematically depicts an infrared (IR) thermometer 10. The thermometer 10 includes a housing 12 including a probe 14 configured to be inserted into a body cavity of a patient. The probe 14 in the illustrated embodiment is configured to be inserted into an ear canal of the patient similar to known infrared medical ear thermometers. The probe 14 could also be similar to the probe 14 of a known medical thermometer that is configured to be placed on the forehead of the patient in the temporal region. Even though the drawings only depict the probe 14 in the shape configured to be inserted into the ear canal, it is to be understood that the probe could take other configurations more conducive to being placed on the forehead of the patient. The thermometer 10 also includes a temperature sensor 16 positioned in the housing 12 and more particularly in the probe 14 in the illustrated embodiment.

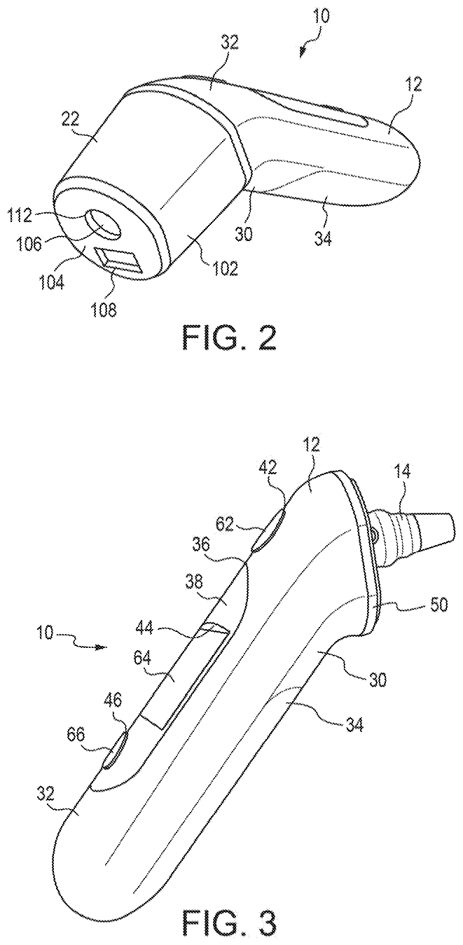

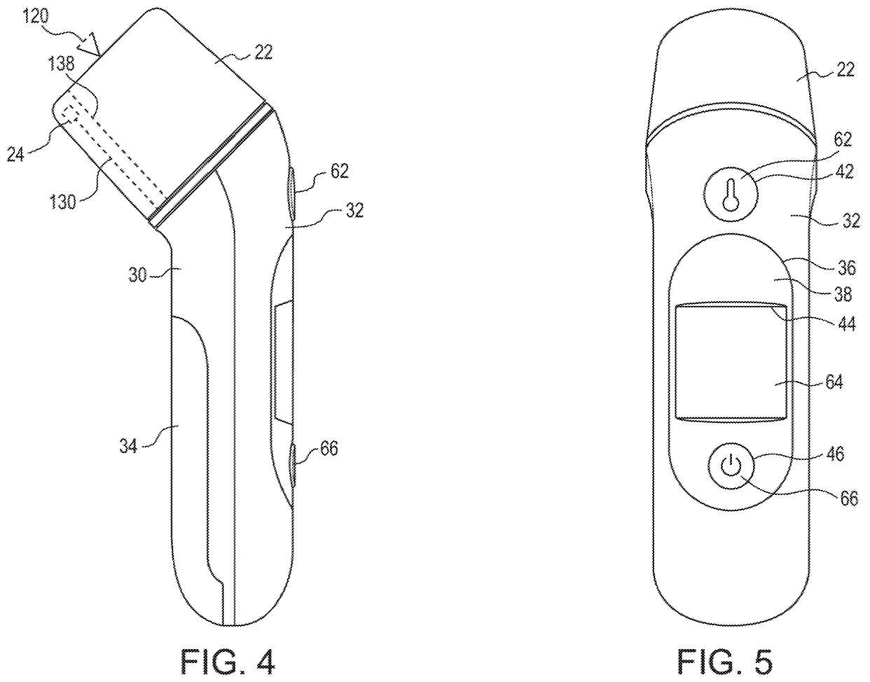

With reference to FIG. 2, the thermometer 10 also includes a cap 22. The cap 22 is configured to cooperate with the housing 12, and is positionable is a first, e.g., a non-contact sensing operating position (shown in FIG. 2), in which the cap 22 is positioned with respect to the probe 14 so as to inhibit insertion of the probe 14 into the body cavity, e.g., ear, of the patient. In the embodiment depicted in FIGS. 2-9, the cap 22 is configured to selectively connect with and to be selectively detached from the housing 12, which can be seen when comparing FIG. 2 to FIG. 3. The cap 22 surrounds and/or covers the probe 14 when connected with the housing 12 with the cap 22 in the non-contact sensing operating position. The thermometer 10 also includes a proximity sensor 24 (depicted schematically in FIG. 4) connected with the cap 22.

With reference back to FIG. 1, the thermometer 10 also includes control circuitry 26 in electrical communication with the temperature sensor 16 and with the proximity sensor 24 when the cap 22 is connected with the housing 12 with the cap 22 in the non-contact sensing operating position. The control circuitry 26 is configured to measure intensity of a first signal, which is received by the temperature sensor 16 and is indicative of the patient's body temperature, and to convert the first signal into a temperature output that reflects a patient's body temperature. The control circuitry 26 is also configured to measure intensity of a second signal, which is received by the proximity sensor 24 and is indicative of the distance between the target area and the proximity sensor 24, and to convert the second signal into a distance output that reflects a distance between the proximity sensor 24 and a target area on the patient's body. The control circuitry 26 is also configured to switch between a first mode and a second mode. The first mode, e.g., a contact mode, is the mode in which the patient's body temperature is being measured with the probe 14 in contact with the patient's body or inserted into the body cavity (e.g., ear canal) of the patient. The second mode, e.g. a non-contact mode, is the mode in which the patient's body temperature is being measured with the cap 22 at least partially covering the probe 14 and the probe and/or the cap 22 being offset from the patient's body. When in the second mode the control circuitry 26 can output a temperature output based on the first (temperature) signal and the second (distance) signal.

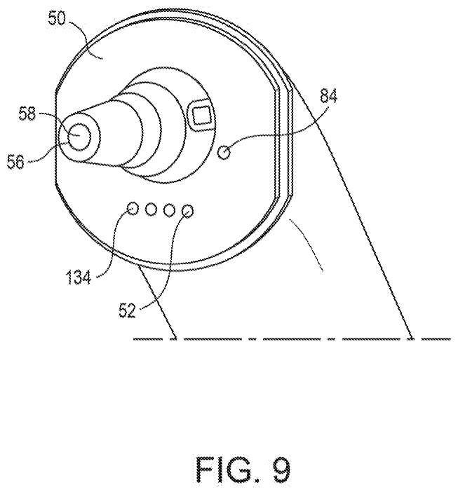

With reference to FIGS. 2 and 3, the housing 12 includes a bottom cover 30 that connects with a top cover 32. The housing 12 also includes a battery cover 34 that is selectively removable from the bottom cover 30. With reference to FIG. 3, the top cover 32 includes a control panel opening 36 (see also FIGS. 5 and 7). A control panel 38 connects with the top cover 32 to cover the control panel opening 36. In the illustrated embodiment, the top cover 32 includes an operation button opening 42 and the control panel 38 includes a display opening 44 and an on/off button opening 46. With reference to FIG. 3, the housing 12 also includes a forward face 50 located at a proximal end of the probe 14. The probe 14 extends forwardly from the forward face 50. With reference to FIG. 9, electrical connector openings 52 are provided in the forward face 50. A temperature sensor opening 56 can be provided at a distal end of the probe 14. A temperature sensor opening window 58 can cover the temperature sensor opening 56.

With reference back to FIG. 3, an operation button 62 connects with the housing 12 and is received in the operation button opening 42. A display 64 also connects with the housing 12 and is provided in the display opening 44 of the control panel 38. An on/off button 66 also connects with the housing 12 and is provided in the on/off button opening 46 provided in the control panel 38. With reference back to FIG. 1, the operation button 62 controls the operation of an operation switch 72 that is in electrical communication with the control circuitry 26. The display 64 is also in electrical communication with the control circuitry 26. The on/off button 66 controls the operation of an on/off switch 76 that is in electrical communication with the control circuitry 26. The thermometer 10 also includes a power supply, which in the illustrated embodiment is a battery 78, which is also in electrical communication with the control circuitry 26.

With continued reference to FIG. 1, the temperature sensor 16 can be a conventional IR sensor configured to detect a thermal radiation signal, which has been referred to above as the first signal. The temperature sensor 16 is positioned adjacent the distal end of the probe 14 and is aligned with the temperature sensor opening 56 (FIG. 9). The temperature sensor 16 is in electrical communication with the control circuitry 26, which is configured to measure the intensity of the first signal and to convert the first signal into a temperature output that reflects a patient's body temperature. Known computing algorithms can be used to convert the intensity of the first signal into the temperature output that reflects the patient's body temperature based on the body site being used to measure the patient's body temperature. Depending on the body site being measured, the control circuitry 26 converts the first signal in a different manner to output an accurate patient body temperature.

As explained above, the control circuitry 26 is configured to switch between at least two modes based on the body site being measured. When the probe 14 is inserted into a body cavity of the patient, the control circuitry 26 can operate in the contact mode and use known algorithms to convert the first signal, which emanates from an ear canal surface, into a temperature output that reflects the patient's body temperature. When the probe 14 takes the configuration of a known medical thermometer that is configured to be placed on the forehead of the patient, the control circuitry 26 can operate in a contact mode and convert the first signal, which is an infrared signal emanating from a forehead region of the patient, and to convert the first signal into a temperature output that reflects the patient's body temperature.

The proximity sensor 24 is positioned in the cap 22 and is configured to detect a signal that is indicative of a distance between the proximity sensor 24 and the target area on the patient's body. The proximity sensor 24 connects with the cap 22 such that the proximity sensor 24 is selectively connectable with and selectively detachable from the housing 12. When the thermometer 10 is used to measure a patient's temperature in a contact mode manner, e.g., by inserting the probe 14 into the patient's ear or other body cavity or by contacting the patient's forehead, the cap 22 and the proximity sensor 24 are removed from the thermometer 10. The proximity sensor 24 can be similar to the distance sensor unit described in U.S. Pat. No. 7,810,992 B2 which includes an IR radiation emitter and a receiver device. When the cap 22 is connected to the housing 12 in the non-contact sensing operating position (shown in FIGS. 2 and 4), the proximity sensor 24 is in electrical communication with the control circuitry 26 (FIG. 1), which is configured to measure and intensity of the second signal received by the proximity sensor 24 and to convert the second signal into a distance output that reflects the distance between the proximity sensor 18 and the target area on the patient's body. In use, the proximity sensor 24 may only operational when the cap 22 is connected to the housing 12 in the non-contact sensing operating position and the control circuitry 26 is in the non-contact mode, which is the mode in which the patient's body temperature is being measured with the cap 22 at least partially covering the probe 14 and the probe 14 or the cap 22 being offset from the patient's body.

With reference to FIG. 1, the thermometer 10 includes an operation mode switch 82 in electrical communication with the control circuitry 26. In one embodiment, operation of the operation mode switch 82 switches the control circuitry 26 between the contact mode and the non-contact mode. With reference to FIG. 9, the thermometer 10 includes an operation mode switch actuator 84 connected with the housing 12 and extending from the forward face 50. The cap 22 cooperates with the operation mode switch actuator 84 to actuate the operation mode switch 82 when the cap 22 is connected with the housing 12 in the non-contact sensing operating position, such as that shown in FIG. 2.

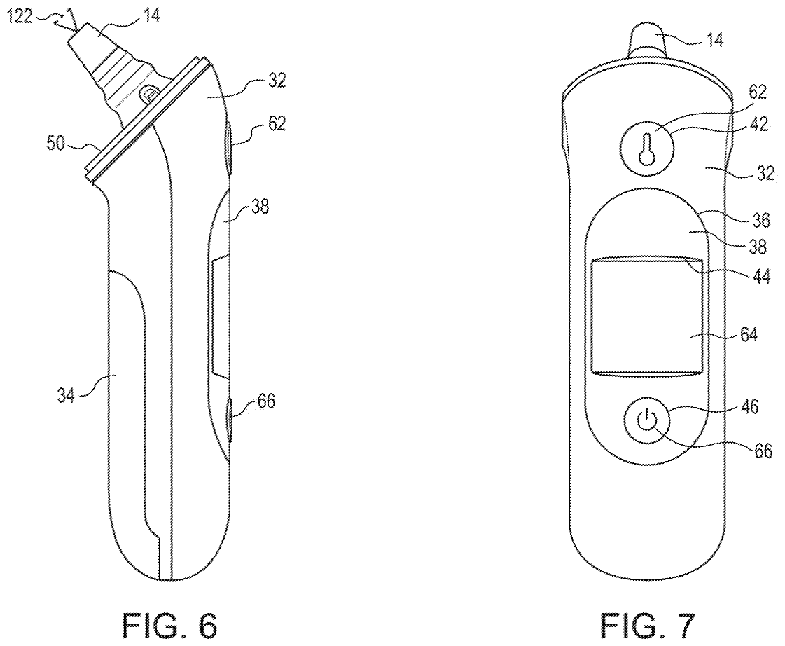

The cap 22 covers, although the cap 22 need not entirely enclose, the probe 14 when the cap 22 is connected with the housing 12 in a non-contact sensing operation position. With reference to FIG. 2, the cap 22 includes a cap side wall 102 and a cap forward wall 104, which can make up a cap housing, made from a material through which light does not pass. The cap side wall 102 is offset from and surrounds the probe 14 when the cap 22 is connected with the housing 12 in the non-contact sensing operating position. The cap forward wall 104 is generally normal to a center axis of the probe 14 when the cap 22 is connected with the housing 12 in the non-contact sensing operating position. A temperature sensor hole 106 and a proximity sensor hole 108 are provided in the cap through at least one of the cap side wall 102 and the cap forward wall 104. The temperature sensor hole 106 is offset from the proximity sensor hole 108. The first signal travels through the temperature sensor hole 106 to be detected by the temperature sensor 16. The second signal travels through the proximity sensor hole 108 to be detected by the proximity sensor 24. A lens 112 can attach to the cap 22 and be located within the temperature sensor hole 106. The lens 112 can be used to alter the field of view of the temperature sensor 16. For example, with reference to FIG. 4, the temperature sensor 16 can have a first field of view 120 (schematically depicted) when the cap 22 is connected to the housing 12. With reference to FIG. 6, the temperature sensor 16 can have a second, different, field of view 122 (schematically depicted) when the cap 22 is not connected to the housing 12. The lens 112 in the temperature sensor hole 106 can be used to change the field of view. Alternatively, other mechanisms, such as light shields, mirrors, and the like, can be used to alter the field of view from when the cap 22 is connected with the housing 12 to when the cap 22 is detached from the housing. These shields and other devices can also be carried by the cap 22.

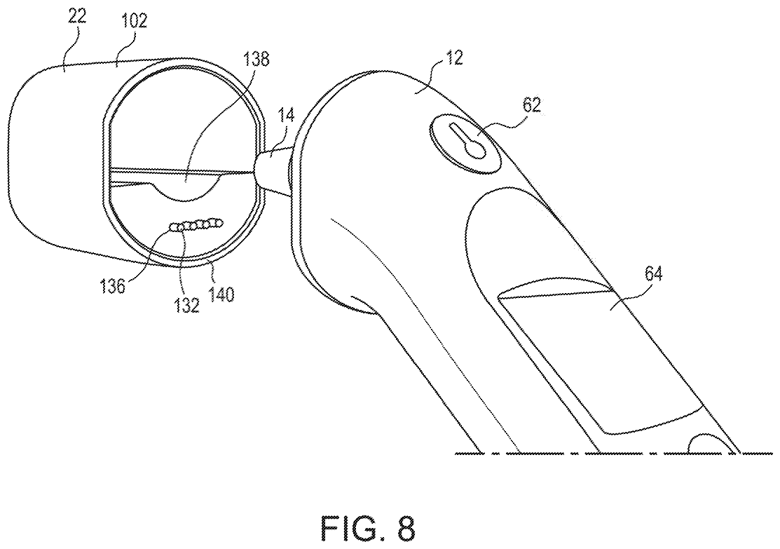

With reference to FIG. 4, the thermometer 10 includes a proximity sensor electrical conductor 130 (depicted schematically) connected with the cap 22 and electrically connected with the proximity sensor 24. The proximity sensor electrical conductor 130 can be one of a plurality of electrical conductors, e.g., wires, located with respect to the cap 22 such that with the cap 22 connected to the housing 12 in the non-contact sensing operating position, the proximity sensor electrical conductor 130 is electrically connected with the control circuitry 26. With reference to FIG. 8, the thermometer 10 includes a first electrical terminal 132, which can be one of a plurality of electrical terminals, electrically connected with the proximity sensor electrical conductor 130. With reference to FIG. 9, the thermometer 10 includes a second electrical terminal 134, which can be one of a plurality of electrical terminals, electrically connected with the control circuitry 26. In the illustrated embodiment, the first electrical terminals 132 are male connector pins that extend through openings 136 provided in a proximity sensor housing 138. The first electrical terminals 132 are positioned adjacent a proximal edge 140 of the cap 22, the proximal edge 140 being positioned adjacent to the housing 12 when the cap 22 is connected with the housing 12 of the thermometer 10. The second electrical terminals 134 are female receptacles aligned with the electrical connector openings 52 provided in the forward face 50 in the illustrated embodiment. The control circuitry 26 can be configured to sense when the first electrical terminal 132 is electrically connected with the second electrical terminal 134 and to switch from the contact mode to the non-contact mode when the first electrical terminal 132 is electrically connected with the second electrical terminal 134, i.e., when the cap 22 is connected with the housing 12 in the non-contact sensing operating position. In such an embodiment, the operation mode switch 82 and the operation mode switch actuator 84 may not be provided.

As mentioned above, the cap 22 includes a proximity sensor housing 138 within the cap 22. The proximity sensor 24 is enclosed by the proximity sensor housing 138 or the cap 22 in combination with the proximity sensor housing 138. The proximity sensor 24 is positioned nearer to the cap forward wall 104 than the proximal edge 140, and in the illustrated embodiment, the proximity sensor 24 is positioned in the cap 22 adjacent to the cap forward wall 104. The proximity sensor housing 138 is positioned adjacent the probe 14 when the cap 22 is connected with the housing 12 in the non-contact sensing operating position so as to preclude a sanitary probe cover (not shown, but similar to the probe cover 6 shown in U.S. Pat. No. 9,591,971 B2) from being connected with the thermometer 10 and covering the probe 14 when the cap 22 is connected with the housing 12 in the non-contact sensing operating position. The aforementioned probe cover is a sanitary envelope that forms a barrier between the probe 14 and the patient. For example, such a sanitary probe cover may be coupled to the thermometer 10 prior to insertion of the thermometer 10 in an ear canal. Alternatively, the cap 22 not in combination with the proximity sensor housing 138 can be configured to preclude a probe cover from being connected with the thermometer 10 and covering the probe 14 when the cap 22 is connected with the housing 12 in the non-contact sensing operating position. Moreover, the cap 22 or the cap 22 in combination with the proximity sensor housing 138 can be configured to allow a probe cover to be connected with the thermometer 10 and covering the probe 14 when the cap 22 is connected with the housing 12 in the non-contact sensing operating position.

In the illustrated embodiment, when the cap 22 is connected with the housing 12 in the non-contact sensing operating position the proximity sensor housing 138 contacts the operation mode switch actuator 84 to activate the operation mode switch 82. When the cap 22 is not connected with the housing 12, the operation mode switch actuator 84 is not activated. This allows the control circuitry 26 to switch from the contact mode when the cap 22 is not connected with the housing 12 to the non-contact mode when the cap 22 is connected with the housing 12 in the non-contact sensing operating position.

The thermometer 10 can be used as a conventional ear thermometer when the probe 14 is inserted into the ear canal of the patient or as a conventional forehead thermometer when the shape of the probe 14 is changed to a more conventional forehead thermometer probe, which is disclosed in U.S. Pat. No. 8,517,603 B2. Operation of the thermometer 10 will be described in more detail with reference to being used as an ear thermometer, however, it should be understood that the operation will be very similar when used as a forehead thermometer when the probe is in contact with the forehead region of the patient. When the thermometer 10 is used as an ear thermometer, the probe 14 is inserted into the ear canal and the operator can push the operation button 62 at which time the temperature sensor 16 detects the first signal which is emanating from an ear canal surface and the control circuitry 26 converts the first signal into an output temperature using a known algorithm. The output temperature can then be displayed on the display 64.

If an operator wants to use the thermometer 10 in a non-contact manner, i.e., a manner in which the probe 14 would not be inserted into an ear canal and the probe 14 would not contact the forehead or other body part of the patient, the operator connects the cap 22 to the housing 12 to cover the probe 14. The cap 22 is connected with the housing 12 in a particular orientation, which is what is referred to as a non-contact sensing operating position, in which the first electrical terminals 132 are inserted into and electrically connected with the second electrical terminals 134. Connecting the cap 22 with the housing 12 also actuates the operation mode switch actuator 84. Actuation of the operation mode switch actuator 84 actuates the operation mode switch 82, which switches the control circuitry 26 from a contact mode to a non-contact mode in which the patient's body temperature is being measured with the cap 22 covering the probe 14 and the cap 22 being offset from the patient's body. The operator can push the operation button 62 to actuate the operation switch 72 at which time the proximity sensor 24 detects the second signal, i.e., a distance signal, and the control circuitry 26 converts the second signal into a distance output that reflects a distance between the proximity sensor 24 and a target area on the patient's body. As discussed above, the proximity sensor 24 can include an IR emitter and receiver device. The thermometer 10 can then be moved forward and backwards with respect to the target area on the patient's body and the proximity sensor 24 and the control circuitry 26 measures the varying distances from the target area until the control circuitry 26 determines that the proximity sensor 24 is at a predetermined distance from the target area at which time the temperature sensor 16 can detect the first signal. Alternatively, an alarm or other operator indication can be provided during the forward and backward movement of the thermometer 10 with respect to the target area and this indication can provide a signal to the operator to again press the operation button 62 at which time the temperature sensor 16 detects the first signal from the target area. By providing the operation mode switch 82, the mode in which the control circuitry 26 displays can be automatic by either appropriately attaching the cap 22 to the housing or having the cap 22 removed from the housing. The operation mode switch can also be located elsewhere and manually operated, if desired, or the control circuitry 26 can determine when the first electrical terminal 132 is electrically connected with the second electrical terminal 134 to change the operation mode.

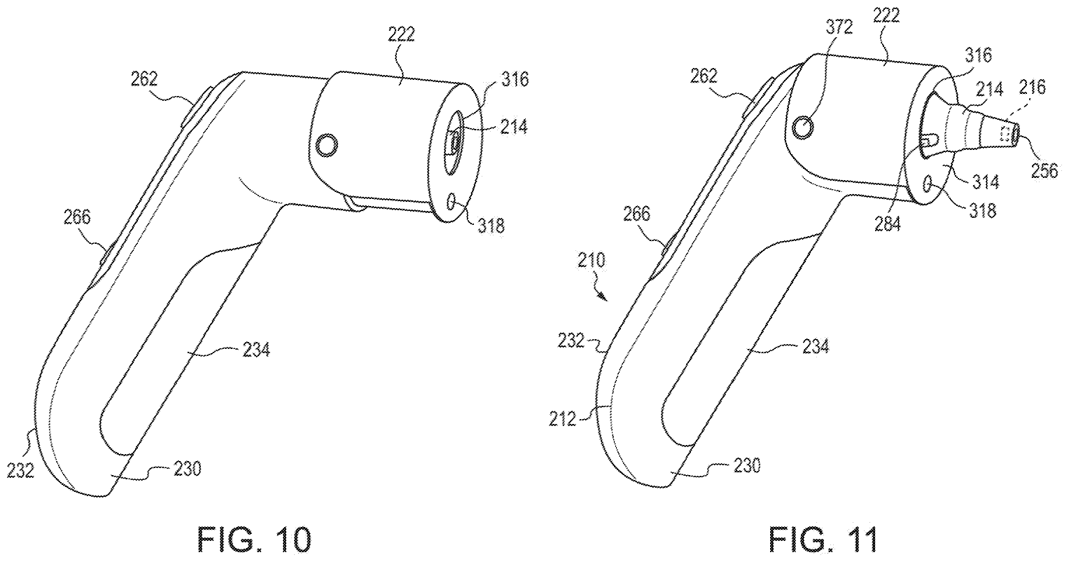

FIGS. 10 and 11 schematically depict an infrared (IR) thermometer 210. The thermometer 210 includes a housing 212 including a probe 214 configured to be inserted into a body cavity of a patient. The probe 214 in the embodiment illustrated in FIGS. 10 and 11 is configured to be inserted into an ear canal of the patient similar to known infrared medical ear thermometers. The thermometer 210 also includes a temperature sensor 216 positioned in the housing 212 and more particularly in the probe 214.

The thermometer 210 also includes a cap 222. The cap 222 is configured to cooperate with the housing 212, and is positionable is a first sensing operating position, e.g., a non-contact sensing operating position (shown in FIG. 10), in which the cap 222 is positioned with respect to the probe 214 so as to inhibit insertion of the probe 214 into the body cavity, e.g., the ear, of the patient. In the embodiment depicted in FIGS. 10 and 11, the cap 222 slides with respect to the probe 214 and the housing 212 between the non-contact sensing operating position (see FIG. 10) and a second sensing operating position, e.g., a contact sensing operating position (see FIG. 11), in which the cap 222 is positioned with respect to the probe 214 so as not to inhibit insertion of the probe into the body cavity of the patient. The cap 222 surrounds and/or covers the probe 214 when in the non-contact sensing operating position. The cap 222 is slid back away from the probe 214 and onto the housing 212 so as to expose the probe 214 when in the contact sensing operating position. The contact sensing operating position shown in FIG. 11 can also be referred to as an in ear mode operating position in which the cap 222 is offset from the probe 214 so as to allow insertion of the probe 214 into the patient's ear. The non-contact sensing operating position shown in FIG. 10 can also be referred to as a forehead mode operating position in which the cap 222 is positioned with respect to the probe 214 so as to inhibit insertion of the probe 214 into a patient's ear. When in the forehead mode operating position, the probe 214 is pointed toward the forehead with the probe 214 offset from the forehead.

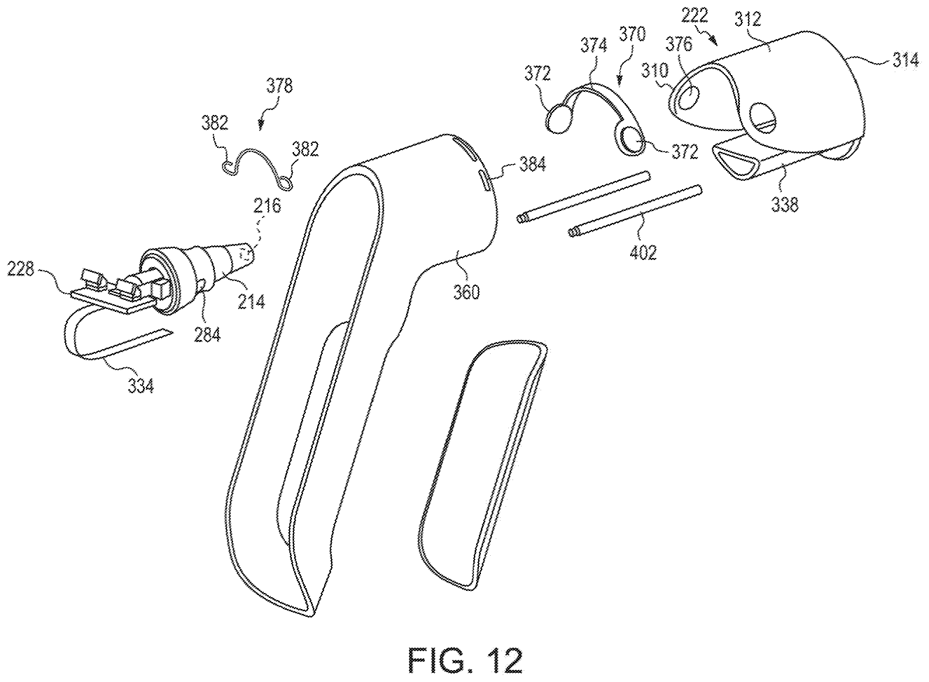

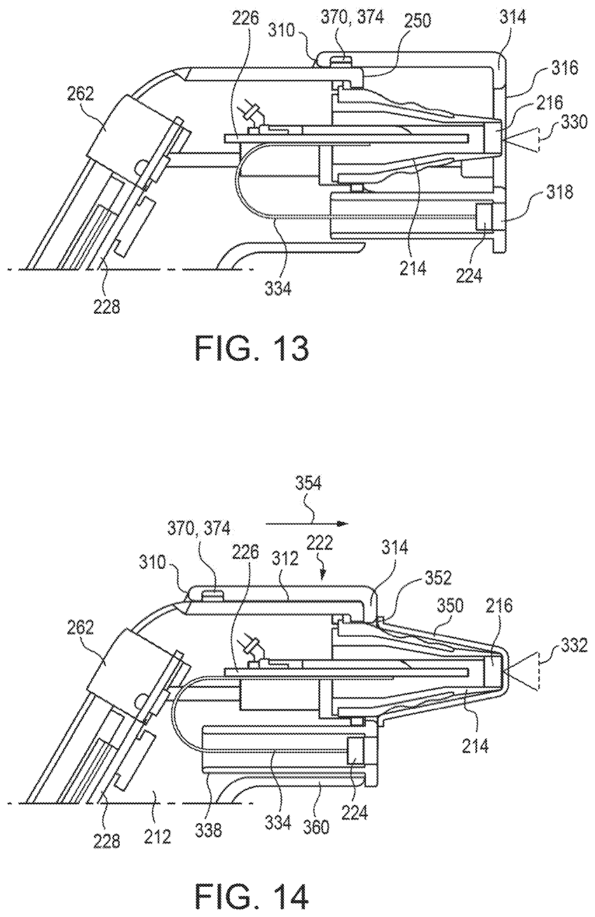

With reference to FIGS. 13 and 14, the thermometer 210 also includes a proximity sensor 224 connected with the cap 222 and control circuitry, which can be provided on or as part of circuit boards 226 and 228 visible in FIGS. 12, 13 and 14. The control circuitry is in electrical communication with the temperature sensor 216 and with the proximity sensor 224. Like the control circuitry 26 described above, the control circuitry is configured to measure intensity of a first signal, which is received by the temperature sensor 216 and is indicative of the patient's body temperature, and to convert the first signal into a temperature output that reflects a patient's body temperature. The control circuitry is also configured to measure intensity of a second signal, which is received by the proximity sensor 224 and is indicative of the distance between the target area and the proximity sensor 224, and to convert the second signal into a distance output that reflects a distance between the proximity sensor 24 and a target area on the patient's body. The control circuitry can also be configured to switch between a first mode, e.g. a contact mode, and a second mode, e.g. a non-contact mode. The contact mode is the mode in which the patient's body temperature is being measured with the probe 214 in contact with the patient's body or inserted into the body cavity (e.g., ear canal) of the patient. The non-contact mode is the mode in which the patient's body temperature is being measured with the cap 222 covering and/or surrounding the probe 214 and the probe 214 being offset from the patient's body.

With reference back to FIGS. 10 and 11, the housing 212 can be similar in many respects to the housing 12 described above. The housing 212 includes a bottom cover 230 that connects with a top cover 232. The housing 212 also includes a battery cover 234 that is selectively removable from the bottom cover 230. With reference to FIG. 13, the housing 12 also includes a forward face 250 located at a proximal end of the probe 214, and the probe 214 extends forwardly from the forward face 250 to a temperature sensor opening 256.

With reference back to FIG. 10, the thermometer can also include an operation button 262, a display (not visible, but similar to the display 64), and an on/off button 66. The operation button 262 controls the operation of an operation switch (similar to the operation switch 72) that is in electrical communication with the control circuitry. An on/off button 266 controls the operation of an on/off switch (similar to the on/off switch 76) that is in electrical communication with the control circuitry. The thermometer 10 also includes a power supply, which can be a battery, which is also in electrical communication with the control circuitry.

With continued reference to FIG. 1, the temperature sensor 216 can be a conventional IR sensor configured to detect a thermal radiation signal, which has been referred to above as the first signal. The temperature sensor 216 is positioned adjacent the distal end of the probe 214 and is aligned with the temperature sensor opening 256. The temperature sensor 216 is in electrical communication with the control circuitry, which is configured to measure the intensity of the first signal and to convert the first signal into a temperature output that reflects a patient's body temperature. Depending on the body site being measured, the control circuitry converts the first signal in a different manner to output an accurate patient body temperature in a similar manner to that described above.

Like the control circuitry 26 described above, the control circuitry in the thermometer 210 is configured to switch between at least two modes based on the body site being measured. When the probe 214 is inserted into a body cavity of the patient, the control circuitry can operate in the contact mode and use known algorithms to convert the first signal, which emanates from an ear canal surface, into a temperature output that reflects the patient's body temperature.

The proximity sensor 224 is positioned in the cap 222 and is configured to detect a signal that is indicative of a distance between the proximity sensor 224 and the target area on the patient's body. The proximity sensor 224 connects with the cap 222 such that the proximity sensor 224 moves with the cap 222 and is selectively movable with respect to the housing 212. The proximity sensor 224 can be similar to the distance sensor unit described in U.S. Pat. No. 7,810,992 B2. When the cap 222 is in the non-contact sensing operating position (shown in FIGS. 10 and 13), the proximity sensor 224 is in electrical communication with the control circuitry, which is configured to measure an intensity of the second signal received by the proximity sensor 224 and to convert the second signal into a distance output that reflects the distance between the proximity sensor 224 and the target area, e.g., the forehead, on the patient's body.

The thermometer 210 can also include an operation mode switch, which is similar in operation to the operation mode switch 82 described above, in electrical communication with the control circuitry. In one embodiment, operation of the operation mode switch switches the control circuitry between the contact mode and the non-contact mode. With reference to FIG. 12, the thermometer 210 can include an operation mode switch actuator 284 connected with the probe 214. Alternatively, the operation mode switch actuator 284 can connect with the housing 212 and extend from the forward face 250, for example. The cap 222 cooperates with the operation mode switch actuator 284 to actuate the operation mode switch when the cap 22 is in the contact sensing operating position, such as that shown in FIG. 11.

The cap 222 covers, although the cap 222 need not entirely enclose, the probe 214 when the cap 222 is in the non-contact sensing operation position. With reference to FIG. 12, the cap 22 includes a proximal edge 310, a cap upper side wall 312 and a cap forward wall 314, which is at an end of the cap upper side wall 312 opposite the proximal edge 310. The cap 222 is made from a material through which light does not pass. The cap upper side wall 312 is offset from and covers the probe 214 when the cap 222 is in the non-contact sensing operating position. The cap forward wall 314 is generally normal to a center axis of the probe 214. In the illustrated embodiment, when the cap 222 is in the contact sensing operating position (FIG. 11) the cap forward wall 314 contacts the operation mode switch actuator 284 to activate the operation mode switch. When the cap 222 is in the non-contact sensing operating position (FIG. 10), the cap forward wall 314 is offset from the operation mode switch actuator 84 such that the operation mode switch actuator 84 is not activated. This allows the control circuitry to switch from the contact mode to the non-contact mode.

A temperature sensor hole 316 and a proximity sensor hole 318 are provided in the cap forward wall 314. The temperature sensor hole 316 is offset from the proximity sensor hole 318. The first signal travels through the temperature sensor hole 316 to be detected by the temperature sensor 216. The second signal travels through the proximity sensor hole 318 to be detected by the proximity sensor 224. With reference to FIG. 13, the temperature sensor 216 can have a first field of view 330 (schematically depicted) when the cap 222 is in the non-contact sensing operation position. With reference to FIG. 14, the temperature sensor 216 can have a second, different, field of view 332 (schematically depicted) when the cap 222 the contact sensing operating position. Mechanisms, such as a lens, light shields, mirrors, and the like, can be used to alter the field of view from when the cap 222 is in the contact sensing operation position to when the cap 222 is in the non-contact sensing operation position. These lenses, light shields, mirrors and other devices can also be carried by the cap 222.

With reference to FIGS. 12 and 13, the thermometer 210 includes a proximity sensor electrical conductor 334 connected with the cap 222 through the proximity sensor 224 and electrically connected with the proximity sensor 224. The proximity sensor electrical conductor 334 can be flexible conductor having slack when in the cap 222 is in the contact sensing operating position. For example, the proximity sensor electrical conductor 334 can be a ribbon cable folded over itself to allow for slack when the cap 222 is in the contact sensing operating position to allow for sliding the cap 222 with respect to the housing 212 toward the non-contact sensing operating position.

The proximity sensor 224 connects with the cap 222 such that the proximity sensor 224 moves with the cap 222, i.e., movement of the cap 222 with respect to the housing 212 results in movement of the proximity sensor 224 with respect to the housing 212. With reference to FIG. 12, the cap 22 includes a proximity sensor housing 338 within the cap 222. The proximity sensor 224 is enclosed by a proximity sensor housing 338, which is provided as part of the cap 222 and is hollow structure that extends rearwardly away from the cap forward wall 314. The proximity sensor 224 is positioned nearer to the cap forward wall 314 than the proximal edge 310, and in the illustrated embodiment, the proximity sensor 224 is positioned in the cap 222 adjacent to the cap forward wall 314. The proximity sensor housing 338 is positioned adjacent the probe 214 when the cap 222 is in the non-contact sensing operating position so as to preclude a sanitary probe cover 350 (shown in FIG. 14 and similar to the probe cover 6 shown in U.S. Pat. No. 9,591,971 B2) from being connected with the thermometer 210 and covering the probe 214 when the cap 222 is in the non-contact sensing operating position.

The cap 222 is positioned with respect to the probe 214 when the cap 222 is in the non-contact sensing operating position so as to preclude the sanitary probe cover 350 (see FIG. 14) from being connected with the thermometer 210 and covering the probe 214 when the cap 222 is in the non-contact sensing operating position. As mentioned above, the sanitary probe cover 350 is a sanitary envelope that forms a barrier between the probe 214 and the patient. The cap 222 can also include a probe cover engagement member 352, which in the illustrated embodiment is a forward surface on the cap forward wall 314. The probe cover engagement member 352 is configured to engage the probe cover 350 covering the probe 214 and to eject the probe cover 350 from the probe 214 when moving from the contact sensing operating position toward the non-contact sensing operating position, e.g., in the direction of arrow 354 in FIG. 14.

With continued reference to FIGS. 12 and 13, the housing 212 includes an upper forward end portion 360 which is configured to allow the cap 222 to move with respect to the housing 212 back and forth in a linear direction parallel to the arrow 354 between the non-contact sensing operating position (see FIG. 13) and the contact sensing operating position (FIG. 14). With reference to FIG. 14, at least a portion of the cap upper side wall 312 is positioned on an exterior of the housing 212 when the cap 222 is in the contact sensing operating position, e.g., the in ear mode operating position. Also, the proximity sensor housing 338 is at least partially received in the housing 212, and more particularly within the upper forward end portion 360, when the cap 222 is in the contact sensing operating position.

With reference to FIG. 12, the thermometer 210 includes a cap latching mechanism configured to cooperate with the cap 222 and the housing 212 to selectively preclude movement of the cap 222 with respect to the housing 212 when the cap 222 is in the non-contact sensing operating position and when the cap is in the contact sensing operating position. The cap latching mechanism includes a button element 370 having a button or trigger (two buttons 372 are provided in the illustrated embodiment) accessible from an exterior of the cap 222. Actuating, e.g., depressing, the buttons 372 allows movement of the cap 222 with respect to the housing 212. The button element 370 includes an arched connector 374 having the buttons 372 provided at each end. Each button 372 extends through a button hole 376 provided in the cap upper side wall 312 adjacent the proximal edge 310 of the cap 222. The buttons 372 cooperate with a spring, which is in the form of a spring clip 378, that biases the buttons 372 outwardly. Ends 382 of the spring clip 378 are received in slots 384 that extend through the housing 212 in the upper forward end portion 360.

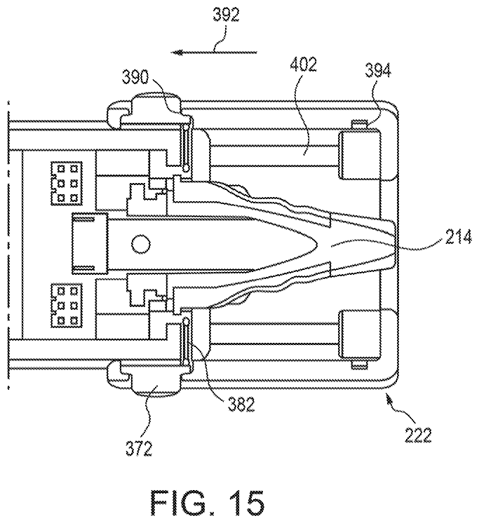

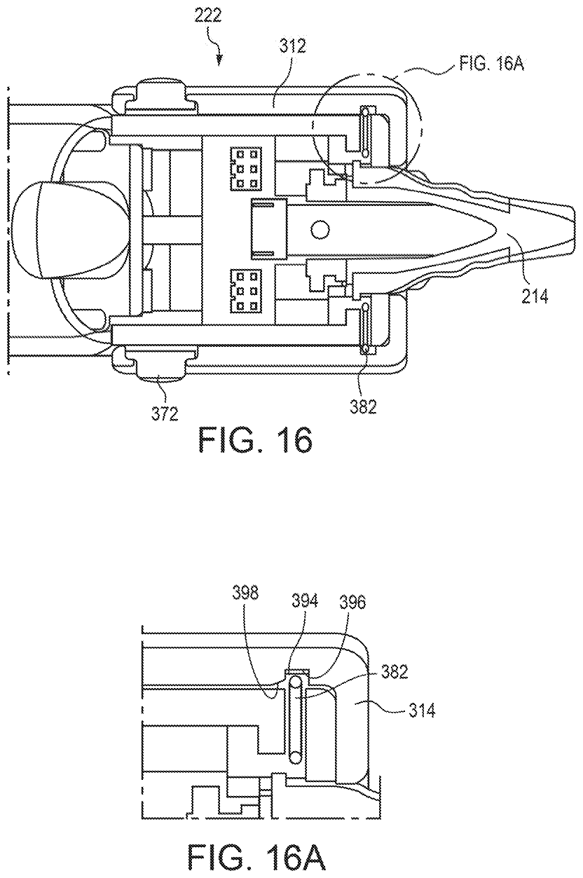

With reference to FIGS. 15 and 16, the ends 382 of the spring clip 378 engage a proximal detent surface 390 when the cap 222 is in the non-contact sensing operating position. Depressing the buttons 372 moves the ends 382 of the spring clip 378 away from proximal detent surface 390, which allows the cap 222 to be slid with respect to the housing 212 in the direction of arrow 392. The ends 382 of the spring clip 378 engage a distal detent surface 394 when the cap 222 is in the contact sensing operating position. The distal detent surface 394 can be provided as part of a distal notch 396 near the cap forward wall 314, and a ramped surface 398 could be provided which urges the ends 382 of the spring clip 378 inward away from the distal detent surface 394, which allows the cap 222 to be slid with respect to the housing 212 in the direction of the arrow 354 (FIG. 14) when the biasing force of the spring clip 378 is overcome. The buttons 372 could also be reconfigured, e.g., by adding an extension that extends toward the cap forward wall 314, such that depressing the buttons 372 moves the ends 382 of the spring clip 378 away from distal detent surface 394, which allows the cap 222 to be slid in the direction of arrow 354. Rods 402 can connect with the cap 222 and the housing 212 to provide support to the cap 222 as it slides with respect to the housing 212.

A method of operating the thermometer 210 will be described, although the method could be accomplished using a thermometer having a configuration different than that shown in FIGS. 10-16. In operation an operator positions the cap 222 in a forehead mode operating position in which the cap 222 is positioned with respect to the probe 214 so as to inhibit insertion of the probe 214 into a patient's ear, which is shown in FIG. 10. With the cap 222 positioned in the forehead mode, the operator points the temperature sensor 216 toward the forehead of the patient and prompts the thermometer 210 to detect a temperature signal, which is indicative of the patient's body temperature, via the temperature sensor 216 and to detect a distance signal, which is indicative of a distance between the proximity sensor 224 and the patient's forehead, via the proximity sensor 224. The operator can prompt the thermometer 210 to detect a temperature signal and the distance signal by depressing the operation button 262, for example. The operator can also position the cap 222 in an in ear mode operating position in which the cap 222 is offset from the probe 214 so as to allow insertion of the probe into the patient's ear, which is shown in FIG. 11. With the cap 222 in the in ear mode, the operator can prompt the thermometer 210 to detect another temperature signal via the temperature sensor 216, e.g., by pushing the operation button 262.

As such, a two-in-one thermometer is provided that can operate in either a contact mode or a non-contact mode. Modifications and alterations of the thermometer may become apparent to those skilled in the art after reading and understanding the preceding detailed description. All such modifications and alterations are intended to be encompassed by the appended claims and the equivalents thereof.

1. A thermometer comprising:
a housing including a probe configured to be placed in contact with a patient's body or to be inserted into a body cavity of the patient;
a temperature sensor positioned in the housing and configured to detect a first signal;
a cap configured to cooperate with the housing, the cap being positionable in a first sensing operating position in which the cap is positioned with respect to the probe so as to inhibit insertion of the probe into the body cavity of the patient;
a proximity sensor connected with the cap, the proximity sensor being configured to detect a second signal; and
control circuitry in electrical communication with the temperature sensor and the proximity sensor, the control circuitry being configured
to measure intensity of the first signal received by the temperature sensor and convert the first signal into a temperature output that reflects a patient's body temperature,
to measure intensity of the second signal received by the proximity sensor and convert the second signal into a distance output that reflects a distance between the proximity sensor and a target area on the patient's body, and
to switch between a first mode in which the patient's body temperature is being measured with the probe being offset from the patient's body and a second mode in which the patient's body temperature is being measured with the probe in contact with the patient's body or inserted into the body cavity of the patient.

2. The thermometer of claim 1, wherein the cap slides with respect to the probe and the housing between the first sensing operating position and a second sensing operating position in which the cap is positioned with respect to the probe so as not to inhibit insertion of the probe into the body cavity of the patient.

3. The thermometer of claim 2, further comprising a proximity sensor electrical conductor electrically connected with the proximity sensor, wherein the proximity sensor electrical conductor is a flexible conductor having slack when in the cap is in the second sensing operating position.

4. The thermometer of claim 2, wherein the cap includes a probe cover engagement member, the probe cover engagement member being configured to engage an associated sanitary probe cover covering the probe and to eject the sanitary probe cover from the probe when moving from the second sensing operating position toward the first sensing operating position.

5. The thermometer of claim 2, wherein the cap includes a side wall, wherein at least a portion of the side wall is positioned on an exterior of the housing when the cap is in the second sensing operating position.

6. The thermometer of claim 2, wherein the cap includes a proximity sensor housing, wherein the proximity sensor housing is at least partially received in the housing when the cap is in the second sensing operating position.

7. The thermometer of claim 2, further comprising a cap latching mechanism configured to cooperate with the cap and the housing so as to selectively preclude movement of the cap with respect to the housing when the cap is in the first sensing operating position and when the cap is in the second sensing operating position.

8. The thermometer of claim 7, wherein the cap latching mechanism includes a button or trigger accessible from an exterior of the cap, wherein actuation of the button or trigger allows movement of the cap with respect to the housing.

9. The thermometer of claim 1, wherein the cap is configured to preclude a sanitary probe cover from being connected with the thermometer and covering the probe when the cap is connected with the housing in the first sensing operating position.

10. The thermometer of claim 1, wherein the cap is made from a material through which light does not pass and includes a temperature sensor hole and a proximity sensor hole, wherein the temperature sensor hole is aligned with a distal end of the probe when the cap is in the first sensing operating position and the proximity sensor hole is aligned with proximity sensor.

11. The thermometer of claim 1, wherein the cap at least partially covers the probe when in the first sensing operating position.

12. The thermometer of claim 1, further comprising an operation mode switch in electrical communication with the control circuitry, wherein operation of the operation mode switch switches the control circuitry between the first mode and the second mode.

13. The thermometer of claim 12, further comprising an operation mode switch actuator connected with the housing, wherein the cap cooperates with the operation mode switch actuator to actuate the operation mode switch when the cap is in at least one of the first sensing operating position and a second sensing operating position.

14. The thermometer of claim 13, wherein the cap cooperates with the operation mode switch actuator to actuate the operation mode switch when the cap is connected with the housing in the first sensing operating position.

15. The thermometer of claim 13, wherein the cap cooperates with the operation mode switch actuator to actuate the operation mode switch when the cap is in the second sensing operating position.

16. The thermometer of claim 1, wherein the temperature sensor has a first field of view when the cap is connected with the housing in the first sensing operating position and a second field of view that is different from the first field of view when the cap is in a second sensing operating position or not connected to the housing.

17. The thermometer of claim 1, wherein the cap selectively connects with and selectively detaches from the housing, and the cap is connected with the housing when in the first operating position.

18. The thermometer of claim 17, further comprising a first electrical terminal electrically connected with a proximity sensor electrical conductor and a second electrical terminal electrically connected with the control circuitry, wherein the control circuitry is configured to sense when the first electrical terminal is electrically connected with the second electrical terminal and to switch from the second mode to the first mode when the first electrical terminal is electrically connected with the second electrical terminal.

19. A method of operating an infrared thermometer including a housing having a probe configured to be placed in a human ear, a temperature sensor positioned in the housing, a cap configured to cooperate with the housing, a proximity sensor connected with the cap and control circuitry in electrical communication with the temperature sensor and the proximity sensor, the method comprising:
positioning the cap in a forehead mode operating position in which the cap is positioned with respect to the probe so as to inhibit insertion of the probe into a patient's ear;
with the cap positioned in the forehead mode, pointing the temperature sensor toward the forehead of the patient and prompting the thermometer to detect a temperature signal, which is indicative of the patient's body temperature, via the temperature sensor and to detect a distance signal, which is indicative of a distance between the proximity sensor and the patient's forehead, via the proximity sensor;
positioning the cap in an in ear mode operating position in which the cap is offset from the probe so as to allow insertion of the probe into the patient's ear; and
with the cap in the in ear mode, prompting the thermometer to detect another temperature signal via the temperature sensor.

20. The method of claim 19, wherein prompting the thermometer to detect the temperature signal includes pushing an operation button.

21. A cap for a thermometer having a probe configured to be placed in contact with a patient's body or to be inserted into a body cavity of the patient and a temperature sensor, the cap comprising:
a cap housing configured to selectively connect with and to be selectively detached from the thermometer and at least partially surround the probe of the thermometer when connected with the thermometer;
a proximity sensor connected with the cap; and
a proximity sensor electrical conductor connected with the cap and electrically connected with the proximity sensor.

22. The cap of claim 21, wherein the cap is configured to cooperate with the temperature sensor to provide a first field of view when the cap is connected to the thermometer and a second field of view that is different from the first field of view when the cap is not connected with the thermometer.

23. The cap of claim 22, wherein the cap housing is made from a material through which light does not pass and includes a temperature sensor hole and a proximity sensor hole aligned with the proximity sensor.

24. The cap of claim 21, further comprising a first electrical terminal electrically connected with the proximity sensor electrical conductor.

25. The cap of claim 24, wherein the first electrical terminal is positioned adjacent a proximal edge of the housing, the proximal edge being positioned adjacent to the thermometer when the cap is connected with the thermometer.

26. The cap of claim 21, further comprising a proximity sensor housing within the cap housing, wherein the proximity sensor is enclosed by the proximity sensor housing or the cap housing in combination with the proximity sensor housing.

27. The cap of claim 26, further comprising a first electrical terminal electrically connected with the proximity sensor electrical conductor, wherein the first electrical terminal extends away from the proximity sensor housing.
